(19) United States
(12) Patent Application Publication
(10) Pub. No.: US 2016/0106506 A1
(43) Pub. Date: Apr. 21, 2016

(54) HAIR REMOVAL APPARATUS

(71) Applicant: LLLUMINAGE LTD., Yoqneam (IL)

(72) Inventors: Shimon ECKHOUSE, Haifa (IL); Lion FLYASH, Nazareth Illit (IL)

(73) Assignee: Illuminage LTD, Yoqneam (IL)

(21) Appl. No.: 14/893,527

(22) PCT Filed: May 27, 2014
(86) PCT No.: PCT/IL2014/000027

§ 371 Date: Nov. 23, 2015

Related U.S. Application Data
(60) Provisional application No. 61/834,895, filed on Jun. 14, 2013.

(51) Int. Cl. A61B 18/20; A45D 26/00; A61N 5/06

(52) U.S. Cl. CPC A61B 18/203; A61N 5/0617; A45D 26/0009; A61B 2018/00476

(57) ABSTRACT

The present apparatus alleviates the unpleasant sensations of the treatment recipient and of the caregiver caused by the heated and sometimes burned hair shafts residuals and vapors emitted by the heated skin.

### TECHNICAL FIELD

The apparatus is related to the field of skin treatment apparatuses and in particular to apparatuses for hair removal by electromagnetic radiation.

### BACKGROUND

Unwanted hair removal using electromagnetic radiation such as pulses of coherent or incoherent light is known in the art. Hair removal has been achieved by inducing the absorption of light in hair follicles and surrounding skin. The light pulses parameters and in particular pulse duration, pulse energy and spectral composition are selected to destroy the hair follicles in the selected skin area.

Generally, hair removal is performed by shaving and the remaining hair shafts and follicles are further destroyed by application of a certain amount of electromagnetic radiation. The electromagnetic radiation incident on the skin is absorbed by hair shafts and almost always increases the temperature of the hair shafts. The energy carried by the electromagnetic radiation and in particular light pulses affects also the skin segment from which the hair has to be removed. Often, in course of the hair removal treatment the temperature of the hair bulbs and the hair shafts reaches a temperature of approximately 70° C.

In course of hair removal treatment light pulses incident on the skin also could heat the skin to a relatively high temperature that could cause skin redness or even slight skin burns followed by burned skin malodor and vapors caused by the high temperature.

Heated and sometimes burned hair shafts emit smoke and often malodor that disturbs the treatment recipient and the caregiver. This is especially pronounced in hairy areas, particularly characterized by dark thick hair.

### SUMMARY

The present apparatus alleviates the unpleasant sensations of the treatment recipient (and of the caregiver) caused by the heated and sometimes burned hair shafts residuals and vapors emitted by the heated skin. The apparatus neutralizes the unpleasant sensation by emitting into the air a material containing fragrance or other aromatic substances that at least reduce the malodor sensation produced by heated and burned hair shaft sand vapors produced by the heated skin that disturbs the treatment recipient and the caregiver.

The apparatus includes an illumination module with a source of light. In course of operation or even stand-by the source of light emits heat that usually is evacuated by a forced flow of cooling air. Typically, a fan produces a forced flow of cooling air. In one example, the material containing fragrance or other aromatic substances is loaded into a disposable cartridge located at the exhaust opening of the cooling air flow channel. The material could include a fragrance causing a pleasant and tranquilizing sensation to the treatment recipient. The material could also include ingredients absorbing the malodors produced by the heated and burned hair shafts and vapors produced by the heated skin. The material could be introduced into the exhausted air in form of a spray or a gas stream. Introduction of the material could be synchronized with the operation of the apparatus, such that the material is introduced in course of hair removal or skin treatment. Alternatively, the material could be introduced in course of the hair removal or skin treatment, before the hair removal or skin treatment, of after the hair removal or skin treatment. The material could be introduced in bursts or pulses or in a continuous material introduction mode.

In one example, a material distribution device disperses the material in the ambient air. The distribution device could be a liquid spraying device or a gas distribution device. The material could include ingredients absorbing the malodors produced by the heated and burned hair shafts and vapors produced by the heated skin. The material could also include a fragrance causing a pleasant and tranquilizing sensation to the treatment recipient. The distribution device could be part of the source of light or an independent material distribution device. Introduction of the material could be synchronized with the operation of the apparatus, such that the material is introduced in course of hair removal or skin treatment. Alternatively, the material could be introduced in course of the hair removal or skin treatment, before the hair removal or skin treatment, of after the hair removal or skin treatment. The material could be distributed in bursts or pulses or in a continuous mode.

In one example, the fragrance material is in form of a dry fragrance. A material distribution device distributes the material in the ambient air. The heat emitted by the source of light could be used to evaporate the dry fragrance. The dry fragrance could be in form of a solid bar or powder. The dry fragrance material could be distributed by a forced flow of cooling air. The dry fragrance material could be in form and shape of a disposable cartridge located at the exhaust opening of the cooling air flow channel. The dry fragrance material could also include ingredients absorbing the malodors produced by the heated and burned hair shafts and vapors produced by the heated skin. The material could also include a fragrance causing a pleasant and tranquilizing sensation to the treatment recipient. The distribution device could be part of the source of light or an independent material distribution device. Introduction of the material in the ambient air could be synchronized with the operation of the apparatus, such that the material is introduced in course of hair removal or skin treatment. Alternatively, the material could be introduced in the ambient air before the hair removal or skin treatment, after the hair removal or skin treatment, or continuously distributed in the ambient air.

In one example, the apparatus could include a pair of RF electrodes configured to apply RF to the segment of the skin to be treated. The RF electrodes could be located on the frame of the source of light and their operation could be synchronized with the operation of the source of light. Alternatively, RF energy could be applied to the treated segment of the skin before the hair removal or skin treatment or in overlapping in time periods.

### BRIEF DESCRIPTION OF THE FIGURES

FIG. 1 is a schematic illustration of an applicator for personal use for skin treatment according to an example;

FIG. 2 is a schematic cross section of the applicator of FIG. 1;

FIG. 3 is a schematic illustration of a side view of the applicator of FIG. 1;

FIG. 4 is a schematic illustration of an illumination source according to an example; and

FIG. 5 is a schematic illustration of an applicator for personal use for skin treatment according to an example.

### DESCRIPTION

The smoke and/or vapor produced in course of hair removal or skin treatment, causing the malodor, are usually purged by the introduction of “external” fresh air at greater than atmospheric pressure. However, such smoke or vapor purging simply disperses the malodor in the room and reduces the malodor relative concentration, but does not resolve the problem.

The present apparatus includes a material distributing device that distributes a material that could include a fragrance or an aromatic substance. The aromatic substance produces a pleasant smell neutralizing the malodor and tranquilizing the treatment recipient. The material could also include ingredients absorbing the malodor. The material could be distributed in course of the skin treatment, before the skin treatment or after the skin treatment.

In the context of the present description the term “malodors” means unpleasant odors and in particular odors which originate from the skin treatment or hair removal treatment.

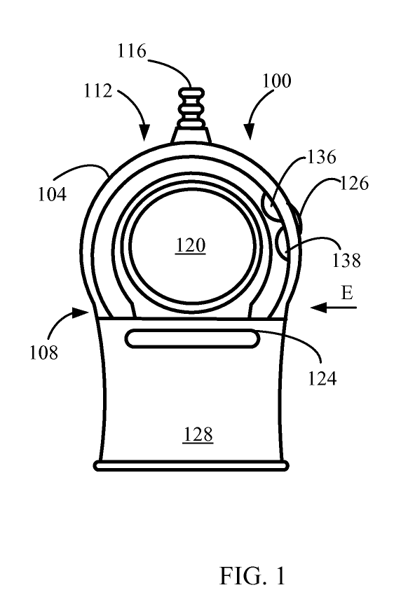

FIG. 1 is a schematic illustration of an applicator for personal use for skin treatment according to an example. Applicator 100 is shown to include an ergonomically designed casing 104, which conveniently fits the hand palm. Casing 104 has a first or distal end 108 and a second end 112, The second end 112 includes a receptacle 116 facilitating connection of applicator 104, with help of a cord, to a docking unit (not shown) or to electrical supply network for charging. A decorative cover 120 covers an air intake opening of the applicator 100. Also shown in the Figure is an ON/OFF switch 126 that could be combined with an indicator informing the user of the operational status of the applicator 100. Alternatively, indicator 124 informing the user of the operational status of the applicator 100 could be a separate indicator.

A first or distal end 108 of applicator 100 includes a receiving bay (not shown) configured to receive an illumination module 128. Module 128 is configured to be applied to a segment of skin to be treated and perform cosmetic skin treatment, which could be just illumination of the skin segment to be treated. The illumination parameters could be selected to perform a desired treatment, which could be for example, hair removal or skin rejuvenation. ON/OFF switch 126 could switch ON or switch OFF skin illumination module 128 when the module is inserted in the receiving bay. Push buttons 136 and 138 could be operated to set different operating parameters of the illumination module 128. The setting of the parameters could be done by a combination of a number of pushes on the buttons 136 and 138.

There could be a number of different illumination modules configured to be used with the same applicator, The different illumination modules could be configured to provide different operational parameters such as light power, wavelength, pulse duration and other operational parameters most suitable for a particular skin treatment or hair removal. For safety reasons electric contacts of each of the modules could be configured to activate electricity supply to illumination module 128 only when the module is inserted into the appropriate receiving bay.

Each of illumination modules 128 could be packed in a cartridge like packaging detachable from the ergonomically designed, fitting the hand casing 104 of applicator 100. The cartridge like packaging of the illumination modules supports use of different illumination modules with the same applicator and easy exchange of the modules.

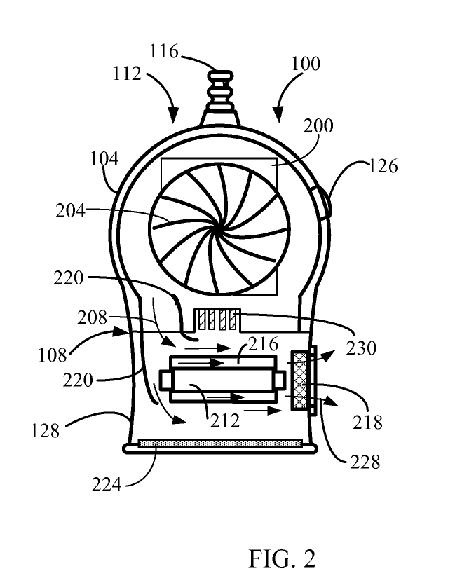

FIG. 2 is an example of a schematic cross section of the applicator of FIG. 1. Case 104 of applicator 100 could be implemented as a two part case and it is shown with one part removed or opened. Case 104 incorporates a blower or a fan 204 providing a stream of cooling air to the illumination module 128, and a control printed circuit board 200. Also shown are a source of light 212 with a reflector 216, and a disposable cartridge 218. Source of light 212 is configured to direct the light emitted by source of light 212 to the treated skin segment. Source of light 212 could direct the light with the help of reflector 216. Alternatively, the internal walls of the illumination module could be coated by a material enhancing light reflection and directing the reflected light to the treated skin segment. Disposable cartridge 218 could contain a certain amount of material including fragrance or aromatic substances. Different air flow directing elements 220 could be introduced into the air flow path to improve the air flow cooling capacity and direct the air flow to specific components of the applicator 100 and/or illumination module 128.

Illumination module 128 could include different source of lights such as an incandescent lamp, xenon lamp, laser diodes, LED, laser or a combination of them. Illumination sources could be configured to operate in a pulsed or continuous operation mode. Their power and operational time could be selected to avoid potential damage to the treated segment of skin.

The protective window 224 located adjacent to the open longitudinal section of reflector 216 forms with the reflector and walls of illumination module 128 a cooling air flowconducting channel. A part of the cooling air flow shown by arrow 222 generated by a cooling fan 204 enters through butt end of reflector 216. The cooling air flow is directed along the air flow conducting channel and along the source of light 212 and cools it The air flow stream as shown by arrows 228 is exhausted through the other butt end opening of reflector 216 and passes through disposable cartridge 218 that contains a certain amount of material including fragrance or aromatic substances. At the exhaust opening of the cooling air flow conducting channel, the temperature of the air flow is elevated, as compared to the temperature of the air flow entering the channel through butt end of reflector 216, and could be between 50 to 70 degrees Celsius. The elevated temperature is sufficient to evaporate some of the fragrance or aromatic substance stored or contained in cartridge 218 and the exhausted air flow 228 distributes the fragrance or aromatic substance in the ambient air. The distributed material containing fragrance or aromatic substance neutralizes or reduces malodor generated in course of hair removal, although continuous distribution of the fragrance or aromatic substance could be performed in a stand by operation mode before the hair removal and after the hair removal. The material containing fragrance or aromatic substance could be impregnated into a fabric or other porous material of which the disposable cartridge 218 could be made. Numeral 230 marks a connector connecting illumination module to control board 200.

In one example, the disposable cartridge could include ingredients absorbing the malodor or made of material that absorbs the malodor. Such material absorbing malodor could be silica gel or activated charcoal. The material absorbing the malodor remains as a part of the disposable cartridge, also in some examples it could be a gas distributed in course of the skin treatment, before the skin treatment or after the skin treatment. In one example, the material could be a combination or mixture of the material including fragrance or aromatic substance and material absorbing the malodor.

Illumination module 128 could include an identifier such as for example, an RFID circuit configured to inform the user on type of the skin (fair or dark) illumination module inserted in the applicator 100. In one example, it could inform the user of the type of treatment for which the illumination module is intended (For example hair removal, skin rejuvenation, acne treatment, and etc.). This could be done by lighting an indicator or sounding a specific sound. In another example, the controller can operate the module with no indication at all as to the type of treatment. The Illumination module will operate according to the type of treatment for which the cartridge was dedicated and the settings made on this base (high, medium, or low power) will change automatically according to the illumination module inserted in a way completely transparent to the user.

Control circuit board 200 could include different sensors such as movement direction sensors, accelerometers, impedance sensors, and others as well as control circuitry of micro switches operated by insertion of the illumination module, temperature sensors processing circuits, illumination module tag identification and treatment parameters setting circuit, and others. Alternatively, some of the sensors may be located in appropriate skin treatment modules and their processing circuits may be located on control board 200. At least one optional audio status indicator such as a buzzer signaling to the user the status of skin treatment process parameters may be attached to applicator 100.

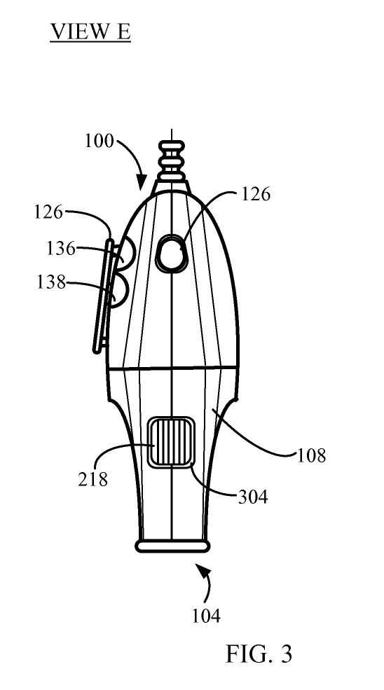

FIG. 3 is a schematic illustration of a side view of an applicator of FIG. 1. It illustrates additional features of the applicator 100 and in particular disposable cartridge 218 that contains the material including fragrance or aromatic substances and/or odor absorbing materials and frame 304. Frame 304 is configured to fix the location of disposable cartridge 218 within illumination module 128 as well as to facilitate a used disposable cartridge 218 extraction and insertion of a new cartridge 218 when required.

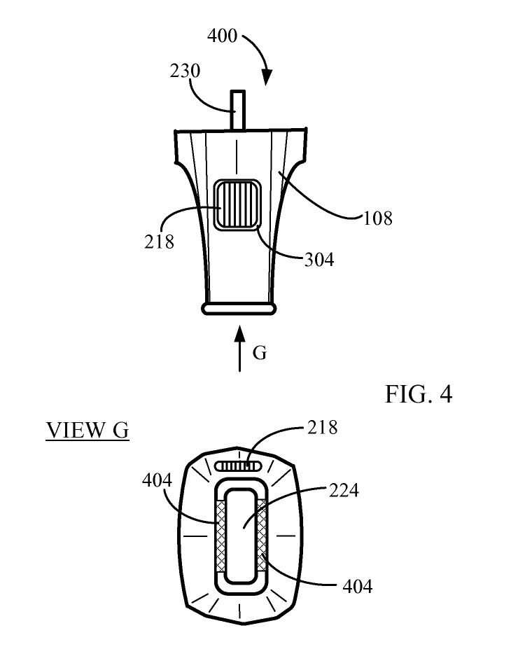

FIG. 4 is a schematic illustration of an illumination source according to an example. Illumination source 400 is similar to illumination source 112. It includes all of the features of illumination source 112 and in addition illumination source 400 includes a pair or RF electrodes configured to apply RF voltage to the treated segment of skin when applicator 100 is applied to the treated segment of skin.

Voltage supplied to RF electrodes could generate current under the skin and heat the skin to a desired temperature and then light emitted by the source of light could be applied to the same segment of the skin. In one example, current caused by the application of RF voltage heats skin volumes located deeper in the skin where hair follicles reside. In such case, the source of light that applies energy to the surface of the skin and in order to heat the hair follicles the energy applied to the surface of the skin has to penetrate deeper skin layers. Combined action of the RF voltage and a source of light could increase faster the temperature of deeper skin layers and support lower surface of the skin temperature and to some extent reduce emission of malodors.

In one example, RF electrodes could be used to determine the treated skin segment temperature. A variable frequency low voltage could be applied to electrodes and impedance of the treated skin segment could be calculated. It is well known that the skin impedance is varies with the skin temperature and accordingly the treated skin segment temperature could be derived.

According to an example, an RFID device is connected to the illumination module. The RFID device could communicate with control board or circuit 200 (FIG. 2). The RFID device could be preloaded with a maximal number of pulses to be emitted before the illumination module 128 has to be replaced and decreases the count with every emitted pulse. Alternatively, the RFID device could be preloaded with a total energy that may be applied to exhaust the cartridge 218 containing the fragrance or aromatic substances.

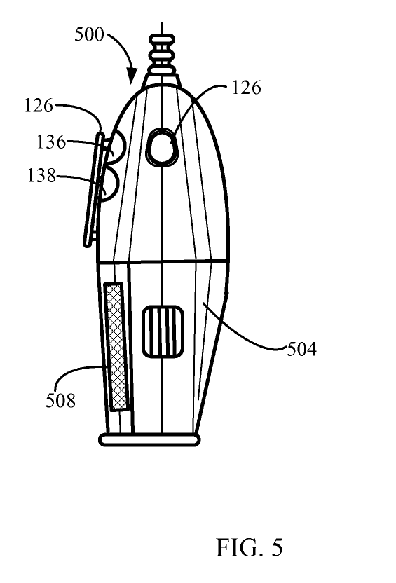

FIG. 5 is a schematic illustration of a side view of an applicator according to an example. Applicator 500 in addition to illumination module 504 could include a separate and independent (autonomous) fragrance or aromatic substances material distribution module 508, which could be a disposable module.

In the earlier examples the cartridge was a passive cartridge and the material containing fragrance or aromatic substance was evaporated or released by the elevated temperature of the cooling air exhaust flow. A separate and independent heater could be used to heat and evaporate the material containing fragrance or aromatic substance located in the cartridge 508. Such cartridge could be termed as an active cartridge. A dedicated fan could be configured to distribute the evaporated material containing fragrance or aromatic substance. The autonomous fragrance or aromatic substance material distribution module could also include a section configured to distribute a material absorbing the malodor.

The materials could be introduced into the exhausted air in form of a spray or a gas stream. Introduction of the materials could be synchronized with the operation of the apparatus, such that the material is introduced in course of hair removal or skin treatment. Alternatively, the material could be introduced in course of the hair removal or skin treatment, before the hair removal or skin treatment, of after the hair removal or skin treatment. The material could be introduced in bursts or pulses or in a continuous mode.

An advantage of such configuration is that the fragrance or aromatic substance as well as material absorbing the malodor could be distributed in bursts or pulses or in a continuous mode. Actually, it can be distributed as it is desired and regulated by the user.

Although the method and apparatus have been described in conjunction with specific examples thereof, it is evident that other alternatives, modifications and variations will be apparent to those skilled in the art Accordingly, it is intended to embrace all such alternatives, modifications and variations that fall within the scope of the appended claims.

What is claimed is:

1. An applicator for hair removal comprising:
at least one source of light configured to be applied to a treated skin segment and illuminate the treated skin segment, said source of light includes at least:
a cooling air flow channel configured to receive a flow of cooling air and cool the source of light and a cooling air flow exhaust opening; and
a cartridge configured to store a material with fragrance or aromatic substance, the cartridge located at the exhaust opening of the cooling air flow channel.

2. The applicator according to claim 1 wherein temperature of the cooling air flow at the exhaust opening of the cooling air flow channel is at elevated temperature as compared to the temperature of the air flow entering the cooling air flow channel through butt end of reflector.

3. The applicator according to claim 1 wherein the cartridge configured to store a material with fragrance or aromatic substance is also configured at elevated temperature to evaporate and release the fragrance or aromatic substance.

4. The applicator according to claim 1 wherein the cooling air flow distributes evaporated fragrance or aromatic substance into ambient air.

5. The applicator according to claim 1 wherein the cooling air flow distributes evaporated material containing fragrance or aromatic substance into ambient air before the hair removal, in course of hair removal, and after the hair removal.

6. The applicator according to claim 1 wherein the cartridge containing material with fragrance or aromatic substance is a disposable cartridge.

7. The applicator according to claim 1 wherein the cartridge with material including fragrance or aromatic substance is one of a group of cartridges consisting of a passive cartridge and an active cartridge.

8. The applicator according to claim 1 wherein the cartridge with material including fragrance or aromatic substance further includes a malodor absorbing material.

9. The applicator according to claim 8 wherein the malodor absorbing material is silica gel or activated charcoal.

10. The applicator according to claim 8 wherein the material including fragrance or aromatic substance is one of a group of materials consisting of liquid material, gas, powder and dry material.

11. An illumination module for an applicator for hair removal comprising:
at least one source of light configured to be applied to a treated skin segment and illuminate the treated skin segment said source of light includes at least:
a cooling air flow channel with an opening configured to receive an air flow and cool the source of light and a cooling air exhaust opening; and
a cartridge configured to store a material with fragrance or aromatic substance, the cartridge located at the cooling air exhaust opening.

12. The illumination module according to claim 11 wherein the cartridge configured to store a material with fragrance or aromatic substance contains fragrance or aromatic substance in liquid form, gas, powder and dry material.

13. The illumination module according to claim 11 wherein the cooling air flow passes through the cartridge configured to store the material with fragrance or aromatic substance and distributes the fragrance or aromatic substance in ambient air.

14. The illumination module according to claim 13 wherein distributed material with fragrance or aromatic substance neutralizes malodor generated in course of hair removal.

15. The illumination module according to claim 11 wherein the cooling air flow distributes the material with fragrance or aromatic substance before the hair removal, in course of hair removal, and after the hair removal.

16. The illumination module according to claim 11 wherein the cartridge configured to store a material with fragrance or aromatic substance includes odor absorbing material.

17. The illumination module according to claim 16 wherein the odor absorbing material is activated charcoal or silica gel.

18. A method for reducing produced in course of hair removal and skin treatment malodor, said method comprising:
applying to an applicator to a segment of skin to be treated the applicator including:
at least one source of light including a cooling air flow channel and a cartridge configured to store a material with fragrance or aromatic substance, said cartridge located at cooling air exhaust opening;
operating an apparatus to remove hair from the segment of skin to be treated; and
distributing fragrance or aromatic substance to neutralize malodor generated in course of hair removal produced by heated and burned hair shafts and vapors produced by heated skin.

19. The method according to claim 18 wherein the fragrance or aromatic substance are supplied in liquid form, gas, powder and solid state.

20. The method according to claim 19 further comprising a separate and independent (autonomous) fragrance or aromatic substances material distribution module.
